(19) United States
(12) Patent Application Publication
(10) Pub. No.: US 2025/0134605 A1
(43) Pub. Date: May 1, 2025

(54) SURGICAL ROBOTIC SYSTEM AND METHOD FOR CONTROLLED TISSUE SEALING

(71) Applicant: Covidien LP, Mansfield, MA (US)

(72) Inventors: Shelby M. Oke, Broomfield, CO (US); Mauri L. Richards, Arvada, CO (US); Jennifer R. Mchenry, Denver, CO (US)

(21) Appl. No.: 18/892,849

(22) Filed: Sep. 23, 2024

Related U.S. Application Data
(60) Provisional application No. 63/593,290, filed on Oct. 26, 2023.

(51) Int. Cl. A61B 34/30; A61B 18/14

(52) U.S. Cl. CPC A61B 34/30; A61B 18/1445; A61B 2034/305

(57) ABSTRACT

A surgical robotic system includes a robotic arm holding an instrument drive unit and a tissue sealing electrosurgical instrument that is actuated by the instrument drive unit to close jaw members to seal tissue. The instrument is an electrosurgical forceps that is also coupled to an electrosurgical generator for energizing the jaw members. The robotic system enters a controlled tissue sealing mode in response to a user input, such as press of a trigger, button, or foot pedal and thereafter automatically controls the electrosurgical generator and the instrument drive unit simultaneously to control energy delivery as well as closing rate and/or pressure of the jaw members, respectively, to form a tissue seal.

### CROSS-REFERENCE TO RELATED APPLICATIONS

This application claims the benefit of, and priority to, U.S. Provisional Patent Application Ser. No. 63/593,290 filed on Oct. 26, 2023. The entire contents of the foregoing application are incorporated by reference herein.

### BACKGROUND

Surgical robotic systems are currently being used in a variety of surgical procedures, including minimally invasive medical procedures. Some surgical robotic systems include a surgeon console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a work site within the patient's body. Surgical robotic systems are used with a variety of jawed surgical instruments, such as graspers, cutters, electrosurgical tissue sealers, etc.

Currently robotically controlled electrosurgical instruments are incapable of automatically operating in a slow closure mode with energy-based sealing devices. During an energy-based sealing process the instrument is energized and then gradually closed by the surgeon, such that the jaws of the device compress and seal tissue in an optimal way. This is very difficult to perform manually as the process is highly technique and energy-dependent due to the number of variables that must be accounted for during this process and lack of haptic feedback in robotic surgery.

### SUMMARY

The present disclosure provides for a controlled slow closure mode for operating robotic electrosurgical tissue sealers, which may be electrosurgical forceps for compressing tissue while applying radio frequency (RF) energy to form a tissue seal (e.g., wedge resections with RF energy, blood vessel sealing, etc.). Since handheld tissue sealers are manually actuated, it is impossible to control the exact rate of closure and force that is applied to the tissue. As a result, it is difficult to impossible to implement the slow close technique using conventional tissue sealers. However, using a surgical robotic system it is possible to control the exact force applied to the tissue sealer as well as the rate of closure. The surgical robotic system may be operated in a controlled (e.g., slow) tissue sealing mode using feedback to determine the degree of compression of the tissue. Suitable feedback parameters include motor torque and position as well as changes in the parameters over time. In addition to monitoring mechanical properties of the tissue, electrical properties are also monitored during the controlled scaling mode. An electrosurgical generator is coupled to the tissue sealer and outputs RF energy using an energy delivery algorithm, during which the generator monitors and reacts to changes in tissue impedance and other energy parameters to determine when the seal is complete. This combination of monitoring and controlling both parameters of the tissue, i.e., mechanical and electrical parameters, allows for repeatability of this tissue sealing technique in a safe and effective way.

According to one embodiment of the present disclosure, a surgical robotic system is disclosed. The system includes a robotic arm having an instrument drive unit and an instrument coupled to and actuatable by the instrument drive unit. The instrument includes a first jaw member and a second jaw member, where one or both of the first jaw member or the second jaw member is movable from an open jaw position to a closed jaw position. The system further includes an electrosurgical generator for outputting electrosurgical energy through the first and second jaw members. The system includes a surgeon console for receiving user input to initiate a tissue sealing mode (e.g., slow scaling mode). The system additionally includes a controller for simultaneously controlling the electrosurgical generator and the instrument drive unit in response to the user input to initiate the tissue sealing mode. The controller commands the electrosurgical generator to output the electrosurgical energy and the instrument drive unit to close one or both of the first jaw member or the second jaw member at a set closure rate to form a tissue seal.

Implementations of the above embodiment may include one or more of the following features. According to one aspect of the above embodiment, the instrument drive unit may include a motor for actuating one or both of the first jaw member or the second jaw member. The instrument drive unit may include a motor sensor for measuring a motor parameter. The motor sensor may be a torque sensor for measuring motor torque. The controller calculates pressure applied by the first and second jaw members based on the motor torque and determines whether the tissue seal is complete based on the calculated pressure. The motor sensor may be a position sensor for measuring angular position of the motor. The controller may calculate a jaw angle between the first and second jaw members based on the angular position of the motor and may also determine whether the tissue seal is complete based on the calculated jaw angle. The electrosurgical generator may include an electrical sensor for measuring a parameter of the electrosurgical energy. The controller may determine whether the tissue seal is complete based on a comparison of the parameter of the electrosurgical energy to a threshold.

According to another embodiment of the present disclosure, a method for controlling a robotically-assisted tissue sealing instrument is disclosed. The method includes receiving user input to start a tissue sealing mode. The method also includes controlling an instrument drive unit in response to the user input to initiate the tissue sealing mode, where the instrument drive unit is coupled to an instrument that includes a first jaw member and a second jaw member, such that one or both of the first jaw member or the second jaw member is movable from an open jaw position to a closed jaw position by the instrument drive unit. The method further includes controlling an electrosurgical generator to output electrosurgical energy through the first and second jaw members in response to the user input to initiate the tissue sealing mode, where the instrument drive unit and the electrosurgical generator are operated simultaneously to compress tissue disposed between the first and second jaw members and supply the electrosurgical energy to the tissue form a tissue seal. The system may use a combination of all of the above, i.e., using the calculated pressure, motor angle, jaw angle, and the generator, to achieve the tissue seal

Implementations of the above embodiment may include one or more of the following features. According to one aspect of the above embodiment, controlling the instrument drive unit may further include controlling a motor for actuating one or both of the first jaw member or the second jaw member. The method may further include measuring a motor parameter at a motor sensor operatively coupled to the motor. Measuring the motor parameter may further include measuring motor torque. The method may also include calculating pressure applied to tissue by the first and second jaw members based on the motor torque and determining whether the tissue seal is complete based on the calculated pressure. Measuring the motor parameter may further include measuring an angular position of the motor. The method may additionally include calculating a jaw angle between the first and second jaw members based on the angular position of the motor and determining whether the tissue seal is complete based on the calculated jaw angle. The method may additionally include measuring at least one parameter of the electrosurgical energy at an electrical sensor operatively coupled to the electrosurgical generator. The method may further include determining whether the tissue seal is complete based on a comparison of the at least one parameter of the electrosurgical energy to a threshold. The method may also include a combination of all of the above, i.e., using the calculated pressure, motor angle, jaw angle, and the generator, to achieve the tissue seal.

According to a further embodiment of the present disclosure, a surgical robotic system is disclosed. The system includes a robotic arm having an instrument drive unit including a motor and a drive sensor for measuring a mechanical parameter, and an instrument coupled to and actuatable by the instrument drive unit. The instrument includes a first jaw member and a second jaw member, where one or both of the first jaw member or the second jaw member is movable from an open jaw position to a closed jaw position. The system also includes an electrosurgical generator for outputting electrosurgical energy through the first and second jaw members. The electrosurgical generator includes a generator sensor for measuring an electrosurgical energy parameter. The system further includes a surgeon console receiving user input to initiate a tissue sealing mode from at least one of a foot pedal, a handle controller, or a screen displaying a graphical user interface. The system additionally includes a controller for simultaneously controlling the electrosurgical generator and the instrument drive unit in response to the user input to initiate the tissue sealing mode, where the controller commands the electrosurgical generator to output the electrosurgical energy and the instrument drive unit to close one or both of the first jaw member or the second jaw member at a set closure rate to form a tissue seal.

Implementations of the above embodiment may include one or more of the following features. According to one aspect of the above embodiment, the controller may further determine whether the tissue seal is formed based on the electrosurgical energy parameter reaching an energy parameter threshold and a mechanical parameter reaching a mechanical threshold.

### BRIEF DESCRIPTION OF THE DRAWINGS

Various embodiments of the present disclosure are described herein with reference to the drawings wherein:

FIG. 1 is a perspective view of a surgical robotic system including a control tower, a console, and one or more surgical robotic arms each disposed on a movable cart according to an embodiment of the present disclosure;

FIG. 2 is a perspective view of a surgical robotic arm of the surgical robotic system of FIG. 1 according to an embodiment of the present disclosure;

FIG. 3 is a perspective view of a movable cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a plan schematic view of movable carts of FIG. 1 positioned about a surgical table according to an aspect of the present disclosure;

FIG. 6 is a perspective view, with parts separated, of an instrument drive unit and a surgical instrument according to an embodiment of the present disclosure;

FIG. 7 is a perspective view of a surgical instrument provided in accordance with the present disclosure configured for mounting on a robotic arm of a robotic surgical system;

FIG. 8 is a front, perspective view of a proximal portion of the surgical instrument of FIG. 7 with an outer shell removed;

FIG. 9 is a rear, perspective view of the proximal portion of the surgical instrument of FIG. 7 with the outer shell removed;

FIG. 10 is a front, perspective view of the proximal portion of the surgical instrument of FIG. 7 with the outer shell and additional internal components removed;

FIG. 11 is a perspective view of a surgical instrument according to another embodiment of the present disclosure;

FIGS. 12A and 12B are perspective views of an end effector of the surgical instrument of FIG. 11 in open and closed configurations;

FIG. 13 is a perspective view of a handle controller according to one embodiment of the present disclosure;

FIG. 14 is a front view of an electrosurgical generator of FIG. 1 according to an embodiment of the present disclosure;

FIG. 15 is a schematic diagram of the electrosurgical generator of FIG. 1 according to an embodiment of the present disclosure;

FIG. 16 is a schematic diagram of a system for determining phases of a surgical procedure according to an embodiment of the present disclosure; and

FIG. 17 is a flow chart of a method for controlled tissue sealing according to an embodiment of the present disclosure.

### DETAILED DESCRIPTION

Embodiments of the presently disclosed surgical robotic system are described in detail with reference to the drawings, in which like reference numerals designate identical or corresponding elements in each of the several views.

As will be described in detail below, the present disclosure is directed to a surgical robotic system, which includes a surgeon console, a control tower, and one or more movable carts having a surgical robotic arm coupled to a setup arm. The surgeon console receives operator input through one or more interface devices. The input is processed by the control tower as movement commands for moving the surgical robotic arm and an instrument and/or camera coupled thereto. Thus, the surgeon console enables teleoperation of the surgical arms and attached instruments/camera. The surgical robotic arm includes a controller, which is configured to process the movement commands to control one or more actuators of the robotic arm, which would, in turn, move the robotic arm and the instrument in response to the movement commands.

The instrument is a forceps having a pair of opposing jaws with one or both of the jaws being movable relative to each other. In embodiments, the forceps may be electrosurgical forceps configured to seal tissue, e.g., blood vessel(s). The jaws are actuated by a drive rod that is engaged by a spring to provide a consistent pressure applied by the opposing jaws on the tissue.

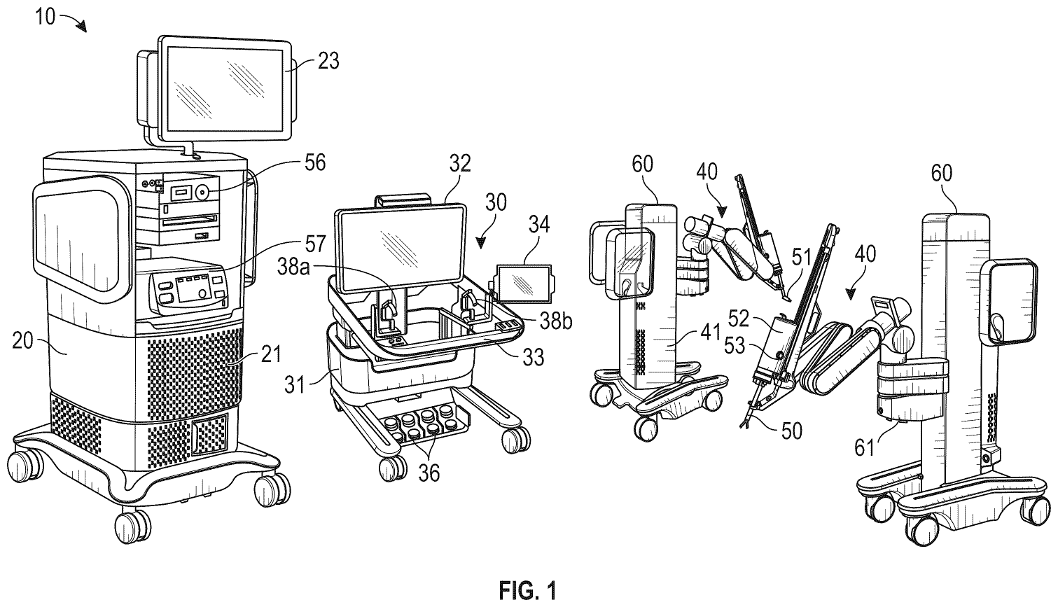

With reference to FIG. 1, a surgical robotic system 10 includes a control tower 20, which is connected to all of the components of the surgical robotic system 10 including a surgeon console 30 and one or more movable carts 60. Each of the movable carts 60 includes a robotic arm 40 having a surgical instrument 50 coupled thereto. The robotic arms 40 also couple to the movable carts 60. The robotic system 10 may include any number of movable carts 60 and/or robotic arms 40.

The surgical instrument 50 is configured for use during minimally invasive surgical procedures. In embodiments, the surgical instrument 50 may be configured for open surgical procedures. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue. In yet further embodiments, the surgical instrument 50 may be a surgical clip applier including a pair of jaws configured apply a surgical clip onto tissue.

One of the robotic arms 40 may include an endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 and output the processed video stream.

The surgeon console 30 includes a first screen 32, which displays a video feed of the surgical site provided by camera 51 disposed on the robotic arm 40, and a second screen 34, which displays a user interface for controlling the surgical robotic system 10. The first screen 32 and second screen 34 may be touchscreens allowing for displaying various graphical user inputs.

The surgeon console 30 also includes a plurality of operator interface devices, such as foot pedals 36 and a pair of handle controllers 38a and 38b which are used by an operator to remotely control robotic arms 40. The surgeon console further includes an armrest 33 used to support clinician's arms while operating the handle controllers 38a and 38b.

The control tower 20 includes a screen 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the handle controllers 38a and 38b. The foot pedals 36 may be used to enable and lock the handle controllers 38a and 38b to reposition the camera and provide for electrosurgical activation/deactivation. In particular, the foot pedals 36 may be used to perform a clutching action on the handle controllers 38a and 38b. Clutching is initiated by pressing one of the foot pedals 36, which disconnects (i.e., prevents movement inputs) the handle controllers 38a and/or 38b from the robotic arm 40 and corresponding instrument 50 or camera 51 attached thereto. This allows the operator to reposition the handle controllers 38a and 38b without moving the robotic arm(s) 40 and the instrument 50 and/or camera 51. This is useful when reaching control boundaries of the surgical space.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

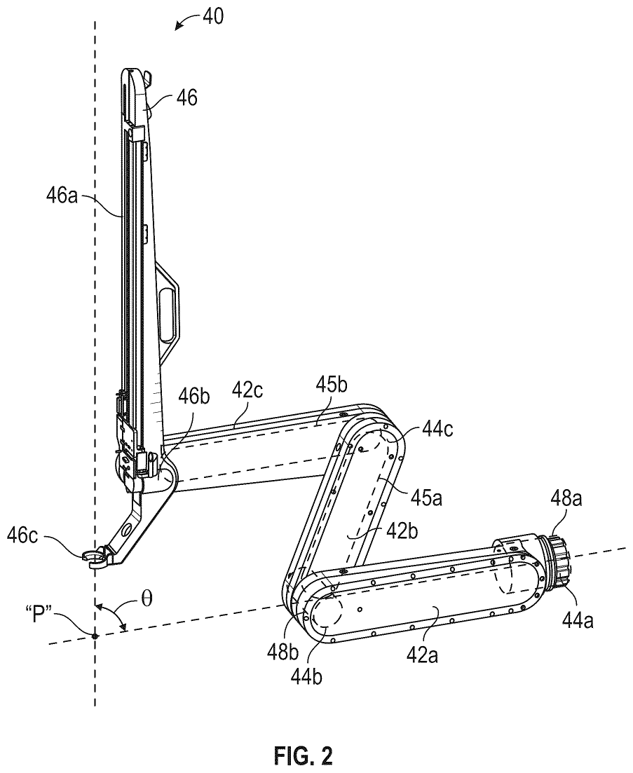

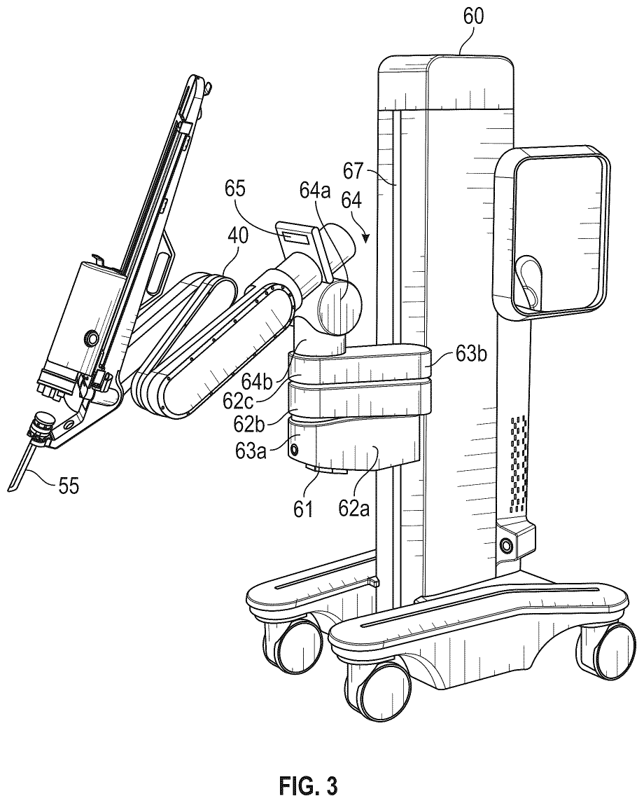

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. Joint 44a is configured to secure the robotic arm 40 to the movable cart 60 and defines a first longitudinal axis. With reference to FIG. 3, the movable cart 60 includes a lift 67 and a setup arm 61, which provides a base for mounting of the robotic arm 40. The lift 67 allows for vertical movement of the setup arm 61. The movable cart 60 also includes a display 65 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components of end effector 140 of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an endoscopic access port 55 (FIG. 3) held by the holder 46. The holder 46 also includes a port latch 46c for securing the access port 55 to the holder 46 (FIG. 2).

The IDU 52 is attached to the holder 46, followed by a sterile interface module (SIM) 43 being attached to a distal portion of the IDU 52. The SIM 43 is configured to secure a sterile drape (not shown) to the IDU 52. The instrument 50 is then attached to the SIM 43. The instrument 50 is then inserted through the access port 55 by moving the IDU 52 along the holder 46. The SIM 43 includes a plurality of drive shafts configured to transmit rotation of individual motors of the IDU 52 to the instrument 50 thereby actuating the instrument 50. In addition, the SIM 43 provides a sterile barrier between the instrument 50 and the other components of robotic arm 40, including the IDU 52.

The robotic arm 40 also includes a plurality of manual override buttons 53 (FIG. 1) disposed on the IDU 52 and the setup arm 61, which may be used in a manual mode. The operator may press one or more of the buttons 53 to move the component associated with the button 53.

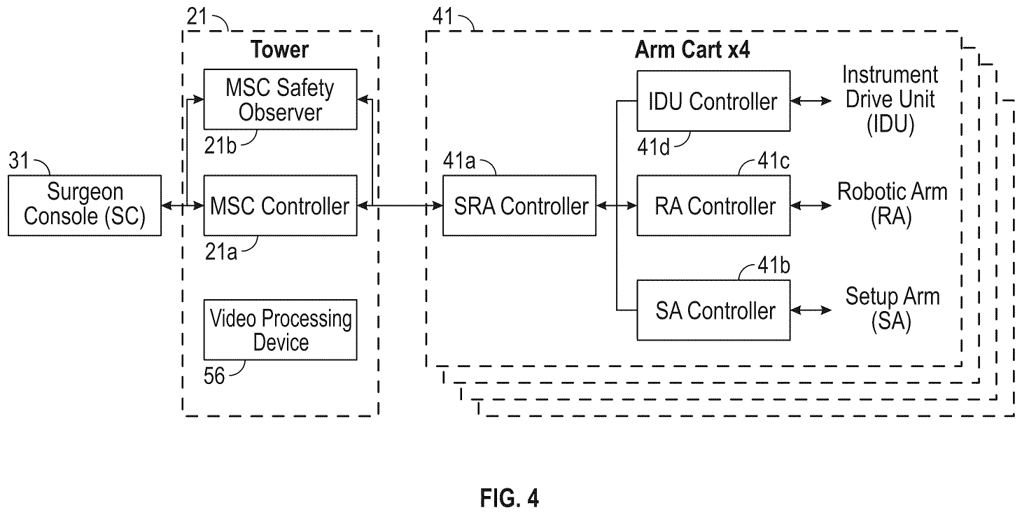

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by an operator. The joints 63a and 63b and the rotatable base 64 include brakes that are disengaged by the operator to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controllers 38a may be embodied as a coordinate position and roll-pitch-yaw (RPY) orientation relative to a coordinate reference frame, which is fixed to the surgeon console 30. The desired pose of the instrument 50 is relative to a fixed frame on the robotic arm 40. The pose of the handle controller 38a is then scaled by a scaling function executed by the controller 21a. In embodiments, the coordinate position may be scaled down and the orientation may be scaled up by the scaling function. In addition, the controller 21a may also execute a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

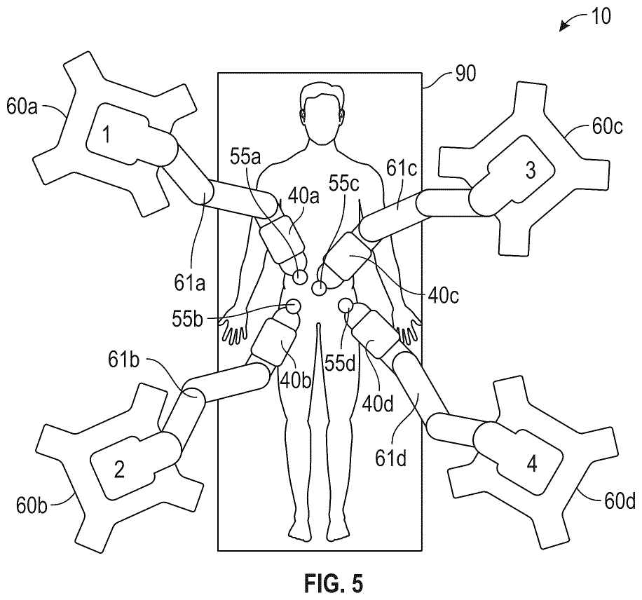

With reference to FIG. 5, the surgical robotic system 10 is set up around a surgical table 90. The system 10 includes movable carts 60a-d, which may be numbered “1” through “4.” During setup, each of the carts 60a-d are positioned around the surgical table 90. Position and orientation of the carts 60a-d depends on a plurality of factors, such as placement of a plurality of access ports 55a-d, which in turn, depends on the surgery being performed. Once the port placements are determined, the access ports 55a-d are inserted into the patient, and carts 60a-d are positioned to insert instruments 50 and the endoscopic camera 51 into corresponding ports 55a-d.

During use, each of the robotic arms 40a-d is attached to one of the access ports 55a-d that is inserted into the patient by attaching the latch 46c (FIG. 2) to the access port 55 (FIG. 3). The IDU 52 is attached to the holder 46, followed by the SIM 43 being attached to a distal portion of the IDU 52. Thereafter, the instrument 50 is attached to the SIM 43. The instrument 50 is then inserted through the access port 55 by moving the IDU 52 along the holder 46.

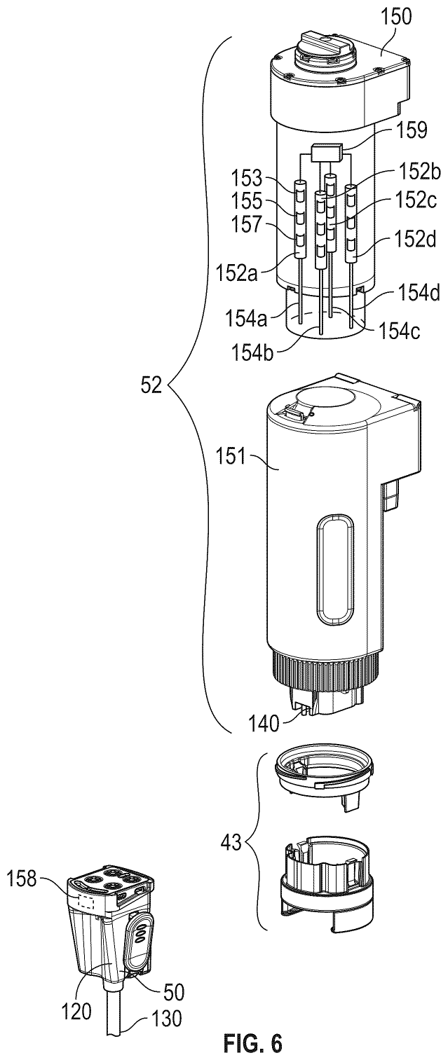

With reference to FIG. 6, the IDU 52 is shown in more detail and is configured to transfer power and actuation forces from its motors 152a-d to the instrument 50 to drive movement of components of the instrument 50, such as articulation, rotation, pitch, yaw, clamping, cutting, etc. The IDU 52 may also be configured for the activation or firing of an electrosurgical energy-based instrument or the like (e.g., cable drives, pulleys, friction wheels, rack and pinion arrangements, etc.).

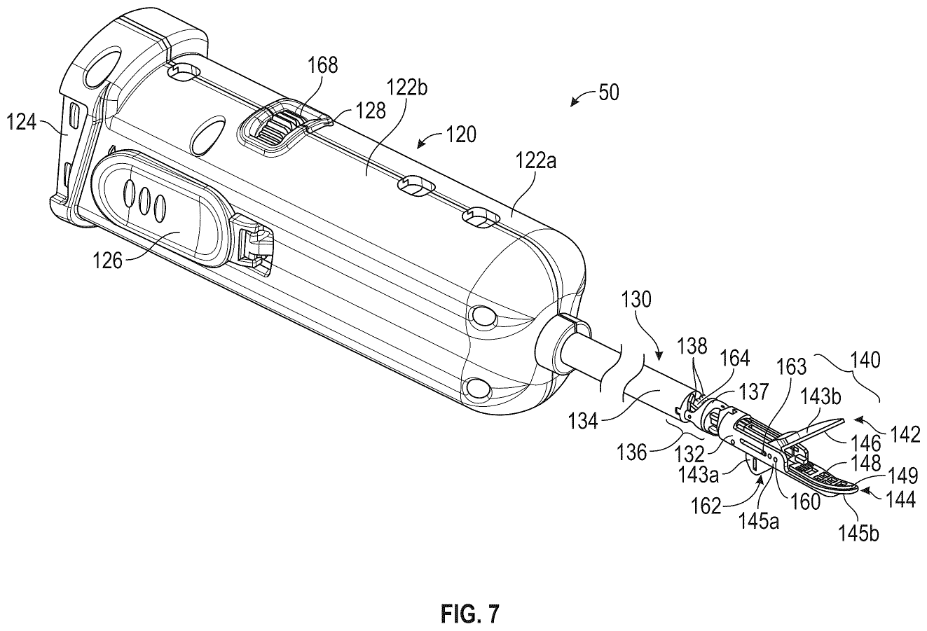

The IDU 52 includes a motor pack 150 and a sterile barrier housing 151. Motor pack 150 includes motors 152a-d for controlling various operations of the instrument 50. The instrument 50 is removably couplable to IDU 52. As the motors 152a-d of the motor pack 150 are actuated, rotation of the drive transfer shafts 154a, 154b, 154c, 154d of the motors 152a-d, respectively, is transferred to the drive assemblies of the instrument 50. The instrument 50 is configured to transfer rotational forces/movement supplied by the IDU 52 (e.g., via the motors 152a-d of the motor pack 150) into longitudinal movement or translation of the cables or drive shafts to effect various functions of an end effector assembly 140 (FIG. 7).

Each of the motors 152a-d includes a current sensor 153, a torque sensor 155, and a position sensor 157, which may be an encoder measuring angular position of the corresponding motor 152a-d. For conciseness only operation of the motor 152a is described below. The sensors 153, 155, 157 monitor the performance of the motor 152a. The current sensor 153 is configured to measure the current draw of the motor 152a and the torque sensor 155 is configured to measure motor torque. The torque sensor 155 may be any force or strain sensor including one or more strain gauges configured to convert mechanical forces and/or strain into a sensor signal indicative of the torque output by the motor 152a. The position sensor 157 may be any device that provides a sensor signal indicative of the number of rotations of the motor 152a, such as a mechanical encoder or an optical encoder. Parameters which are measured and/or determined by the position sensor 157 may include speed, distance, revolutions per minute, position, and the like. The sensor signals from sensors 153, 155, 157 are transmitted to the IDU controller 41d, which then controls the motors 152a-d based on the sensor signals. In particular, the motors 152a-d are controlled by an actuator controller 159, which controls torque output by, and angular velocity of the motors 152a-d. In embodiments, additional position sensors may also be used, which include, but are not limited to, potentiometers coupled to movable components and configured to detect travel distances, Hall Effect sensors, accelerometers, and gyroscopes. In embodiments, a single controller can perform the functionality of the IDU controller 41d and the actuator controller 159.

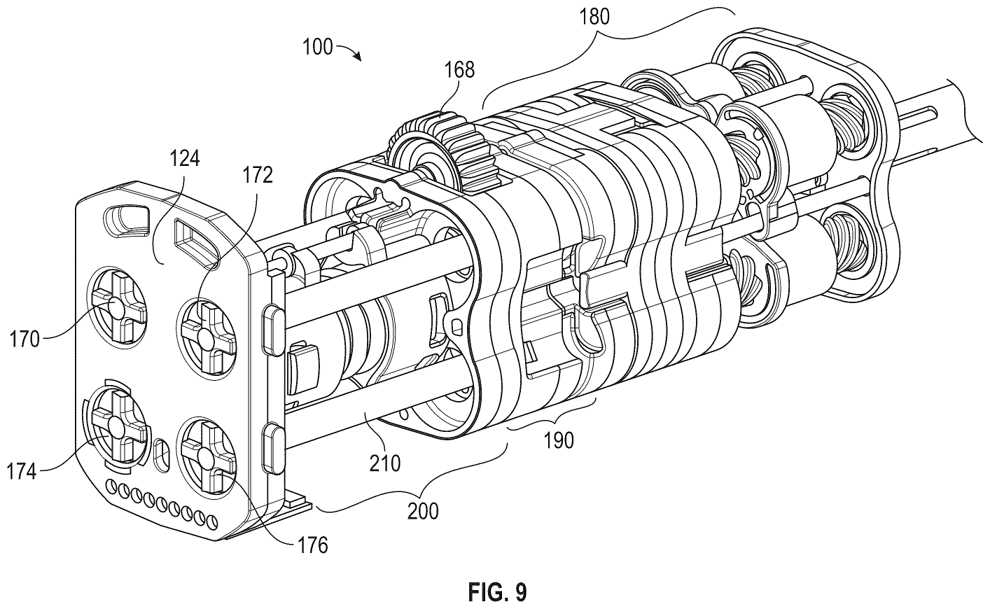

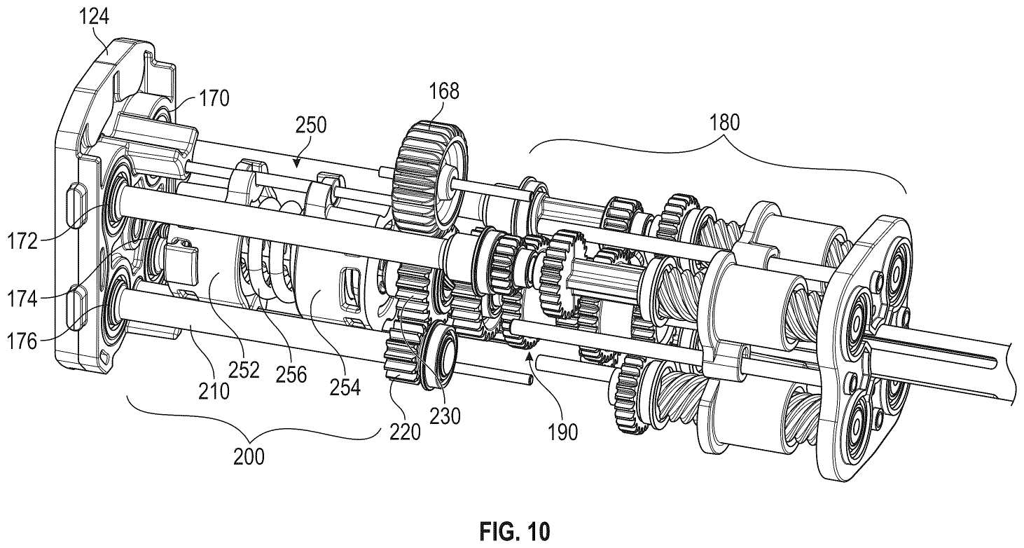

Referring to FIGS. 6-10, the instrument 50 includes the housing 120, a shaft 130 extending distally from housing 120, and end effector assembly 140 extending distally from shaft 130. A gearbox assembly 100 disposed within housing 120 and operably associated with end effector assembly 140. Housing 120 of instrument 50 is configured to selectively couple to IDU 52 of robotic, to enable motors 152a-d of IDU 52 to operate the end effector assembly 140 of the instrument 50. Housing 120 of instrument 50 supports a drive assembly that is mechanically actuated by the motors 152a-d of the IDU 52. The drive assembly of instrument 50 may include any suitable electrical and/or mechanical component to effectuate driving force/movement.

Instrument 50 is described herein as an articulating electrosurgical forceps configured for use with the robotic surgical system 10. However, the aspects and features of instrument 50 provided in accordance with the present disclosure, detailed below, are equally applicable for use with other suitable surgical instruments and/or in other suitable surgical systems.

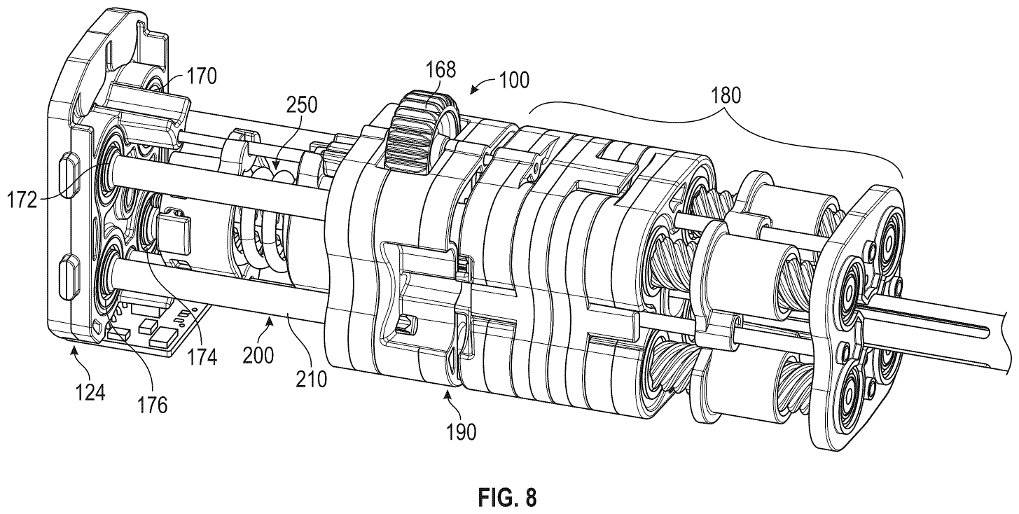

The housing 120 of instrument 50 includes first and second body portion 122a, 122b and a proximal face plate 124 that cooperate to enclose gearbox assembly 100 therein. Proximal face plate 124 includes apertures defined with couplers 170, 172, 174, 176 of gearbox assembly 100 extending through proximal face plate 124 (FIG. 8). A pair of latch levers 126 (only one of which is illustrated in FIG. 7) extend outwardly from opposing sides of housing 120 and enable releasable engagement of housing 120 with the IDU 52 of the robotic arm 40. An aperture 128 defined through housing 120 permits thumbwheel 168 to extend therethrough to enable manual manipulation of thumbwheel 168 from the exterior of housing 120 to permit manual opening and closing of end effector assembly 140.

Shaft 130 of instrument 50 includes a distal segment 132, a proximal segment 134, and an articulating section 136 disposed between the distal and proximal segments 132, 134, respectively. Articulating section 136 includes one or more articulating components 137, e.g., links, joints, etc. A plurality of articulation cables 138, e.g., four (4) articulation cables, or other suitable actuators, extend through articulating section 136. More specifically, articulation cables 138 are operably coupled to distal segment 132 of shaft 130 at the distal ends thereof and extend proximally from distal segment 132 of shaft 130, through articulating section 136 of shaft 130 and proximal segment 134 of shaft 130, and into housing 120, wherein articulation cables 138 operably couple with an articulation sub-assembly 180 of gearbox assembly 100 to enable selective articulation of distal segment 132 (and, thus end effector assembly 140) relative to proximal segment 134 and housing 120, e.g., about at least two axes of articulation (e.g., yaw and pitch articulation). Articulation cables 138 may be arranged in a generally rectangular configuration, although other suitable configurations are also contemplated.

Articulation of end effector assembly 140 relative to proximal segment 134 of shaft 130, is accomplished by actuation of articulation cables 138. More specifically, in order to pitch end effector assembly 140, the upper pair of cables 138 are actuated while the lower pair of cables 138 are actuated relative to one another but in an opposite manner relative to the upper pair of cables 138. With respect to yaw articulation, the right pair of cables 138 are actuated while the left pair of cables 138 are actuated but in an opposite manner relative to the right pair of cables 138.

Continuing with reference to FIG. 7, end effector assembly 140 includes first and second jaw members 142, 144, respectively. Each jaw member 142, 144 includes a proximal flange portion 143a, 145a and a distal body portion 143b, 145b, respectively. Distal body portions 143b, 145b define opposed tissue-contacting surfaces 146, 148, respectively. Proximal flange portions 143a, 145a are pivotably coupled to one another about a pivot 160 and are operably coupled to one another via a cam-slot assembly 162 including a cam pin 163 slidably received within cam slots defined within the proximal flange portion 143a, 145a of at least one of the jaw members 142, 144, respectively, to enable pivoting of jaw member 142 relative to jaw member 144 and distal segment 132 of shaft 130 between a spaced-apart position (e.g., an open position of end effector assembly 140) and an approximated position (e.g. a closed position of end effector assembly 140) for grasping tissue between tissue-contacting surfaces 146, 148. As an alternative to this unilateral configuration, a bilateral configuration may be provided whereby both jaw members 142, 144 are pivotable relative to one another and distal segment 132 of shaft 130.

In embodiments, longitudinally extending knife channels 149 (only knife channel 149 of jaw member 144 is illustrated; the knife channel of jaw member 142 is similarly configured) are defined through tissue-contacting surfaces 146, 148, respectively, of jaw members 142, 144. In such embodiments, a knife assembly including a knife tube (not shown) extending from housing 120 through shaft 130 to end effector assembly 140 and a knife blade (not shown) disposed within end effector assembly 140 between jaw members 142, 144 is provided to enable cutting of tissue grasped between tissue-contacting surfaces 146, 148 of jaw members 142, 144, respectively. Knife tube (not shown) is operably coupled to a knife drive sub-assembly 190 of gearbox assembly 100 at a proximal end thereof to enable selective actuation thereof to, in turn, reciprocate the knife blade (not shown) between jaw members 142, 144 to cut tissue grasped between tissue-contacting surfaces 146, 148.

Referring to FIG. 7, a drive rod 164 is operably coupled to cam-slot assembly 162 of end effector assembly 140, e.g., engaged with the cam pin 163 thereof, such that longitudinal actuation of drive rod 164 pivots jaw member 142 relative to jaw member 144 between the spaced-apart and approximated positions. More specifically, urging drive rod 164 proximally pivots jaw member 142 relative to jaw member 144 towards the approximated or closed position while urging drive rod 164 distally pivots jaw member 142 relative to jaw member 144 towards the spaced-apart or open position. However, other suitable mechanisms and/or configurations for pivoting jaw member 142 relative to jaw member 144 between the spaced-apart and approximated positions in response to selective actuation of drive rod 164 are also contemplated. Drive rod 164 extends proximally from end effector assembly 140 through shaft 130 and into housing 120 wherein drive rod 164 is operably coupled with a jaw drive sub-assembly 200 of gearbox assembly 100 to enable selective actuation of end effector assembly 140 to grasp tissue therebetween and apply a closure force within an appropriate force range.

Tissue-contacting surfaces 146, 148 of jaw members 142, 144, respectively, are at least partially formed from an electrically conductive material and are energizable to different potentials to enable the conduction of electrical energy through tissue grasped therebetween, although tissue-contacting surfaces 146, 148 may alternatively be configured to supply any suitable energy, e.g., thermal, microwave, light, ultrasonic, ultrasound, etc., through tissue grasped therebetween for energy-based tissue treatment. Instrument 50 defines a conductive pathway (not shown) through housing 120 and shaft 130 to end effector assembly 140 that may include lead wires, contacts, and/or electrically-conductive components to enable electrical connection of tissue-contacting surfaces 146, 148 of jaw members 142, 144, respectively, to an energy source (not shown), e.g., an electrosurgical generator, for supplying energy to tissue-contacting surfaces 146, 148 to treat, e.g., seal, tissue grasped between tissue-contacting surfaces 146, 148.

With reference to FIGS. 8-10, the gearbox assembly 100 is disposed within housing 120 and includes an articulation sub-assembly 180, a knife drive sub-assembly 190, and a jaw drive sub-assembly 200. Articulation sub-assembly 180 is operably coupled between first and second couplers 170, 172, respectively, of gearbox assembly 100 and articulation cables 138 (FIG. 7) such that, upon receipt of appropriate inputs into first and/or second couplers 170, 172, articulation sub-assembly 180 manipulates cables 138 (FIG. 7) to articulate end effector assembly 140 in a desired direction, e.g., to pitch and/or yaw end effector assembly 140.

Knife drive sub-assembly 190 is operably coupled between third coupler 174 of gearbox assembly 100 and knife tube (not shown) such that, upon receipt of appropriate input into third coupler 174, knife drive sub-assembly 190 manipulates knife tube to reciprocate the knife blade (not shown) between jaw members 142, 144 to cut tissue grasped between tissue-contacting surfaces 146, 148.

Jaw drive sub-assembly 200, as detailed below, is operably coupled between fourth coupler 176 of gearbox assembly 100 and drive rod 164 such that, upon receipt of appropriate input into fourth coupler 176, jaw drive sub-assembly 200 pivots jaw members 142, 144 between the spaced-apart and approximated positions to grasp tissue therebetween and apply a closure force within an appropriate closure force range.

Gearbox assembly 100 is configured to operably interface with the IDU 52 when instrument 50 is mounted on robotic surgical system 10. That is, the motors 152a-d of IDU 52 selectively actuate couplers 170-176 of gearbox assembly 100 to articulate end effector assembly 140, grasp tissue between jaw members 142, 144, and/or cut tissue grasped between jaw members 142, 144. However, it is also contemplated that gearbox assembly 100 be configured to interface with any other suitable surgical system, e.g., a manual surgical handle, a powered surgical handle, etc.

With reference to FIGS. 8-10, jaw drive sub-assembly 200 of gearbox assembly 100 is shown generally including a drive shaft 210, an input gear 220, a drive gear 230, the thumbwheel 168, a spring force assembly 250, and the drive rod assembly 164. The spring force assembly 250 includes a proximal hub 252, a distal hub 254, and a compression spring 256.

Compression spring 256 is disposed between proximal and distal hubs 252, 254 with a proximal portion thereof disposed within a cavity of proximal hub 252 and a distal portion thereof disposed within a cavity of distal hub 254. At least a portion of compression spring 256 is disposed about and/or configured to receive a portion of lead screw of drive gear 230 therethrough.

During use, jaw members 142, 144 are initially disposed in the spaced-apart position and, correspondingly, proximal and distal hubs 252, 254 are disposed in a distal-most position such drive rod 164 is disposed in a distal-most position. Further, in this position, compression spring 256 is disposed in a least-compressed condition; although even in the least-compressed condition, compression spring 256 may be partially compressed due to the retention of compression spring 256 in a pre-compressed configuration between proximal and distal hubs 252, 254.

In response to an input to close end effector assembly 140, e.g., rotational input to fourth (i.e., jaw) coupler 176 or a manual rotation of the thumbwheel 168, drive shaft 210 is rotated to thereby rotate input gear 220 which, in turn, rotates drive gear 230 such that distal hub 254 is translated proximally towards proximal hub 252. Proximal translation of distal hub 254 urges distal hub 254 against compression spring 256. Initially, where forces resisting approximation of jaw members 142, 144 are below a threshold corresponding to the spring value of compression spring 256, the closure force applied by jaw members 142, 144 is relatively low such that the urging of distal hub 254 proximally against compression spring 256 urges compression spring 256 proximally which, in turn, moves drive rod 164 proximally to pivot jaw member 142 relative to jaw member 144 from the spaced-apart position towards the approximated position to grasp tissue therebetween.

Upon further approximation of jaw members 142, 144 to grasp tissue therebetween, the forces resisting approximation of jaw members 142, 144, e.g., tissue resisting compression, may reach the threshold and, thus the closure force applied by jaw members 142, 144 may reach a corresponding threshold. In order to maintain the closure force applied by jaw members 142, 144 within a closure pressure range such as, for example, from about 3 kg/cm² to about 16 kg/cm², application of further closure force by jaw members 142, 144 is inhibited beyond this point despite further rotational input to fourth coupler 176. Once the threshold has been reached, further rotational input to fourth coupler 176 rotates drive shaft 210, input gear 220, and drive gear 230 to translate distal hub 254 further proximally into compression spring 256. However, rather than compression spring 256 urging proximal hub 252 further proximally to continue approximation of jaw members 142, 144 and increase the closure force applied therebetween, compression spring 256 is compressed, enabling proximal hub 252 and, thus, drive rod 164 to remain in position, thus inhibiting application of additional closure force between jaw members 142, 144. Operation of the instrument 50 and its components, including the compression spring 256, is described in more detail in U.S. Patent Application Publication No. 2020/0237453, filed on Jan. 29, 2019, the entire contents of which are incorporated by reference herein.

The surgical instrument 50 also includes a storage device 158 (FIG. 6). The storage device 158 includes non-volatile storage medium (e.g., EEPROM) that is configured to store any data pertaining to the surgical instrument 50, including but not limited to, usage count, identification information, model number, serial number, calibration data, and the like. In embodiments, the data may be encrypted and is only decryptable by the IDU controller 41d. The data may also be used by the IDU controller 41d to authenticate the surgical instrument 50. The storage device 158 may be configured in read only or read/write modes, allowing the IDU controller 41d to read as well as write data onto the storage device 158.

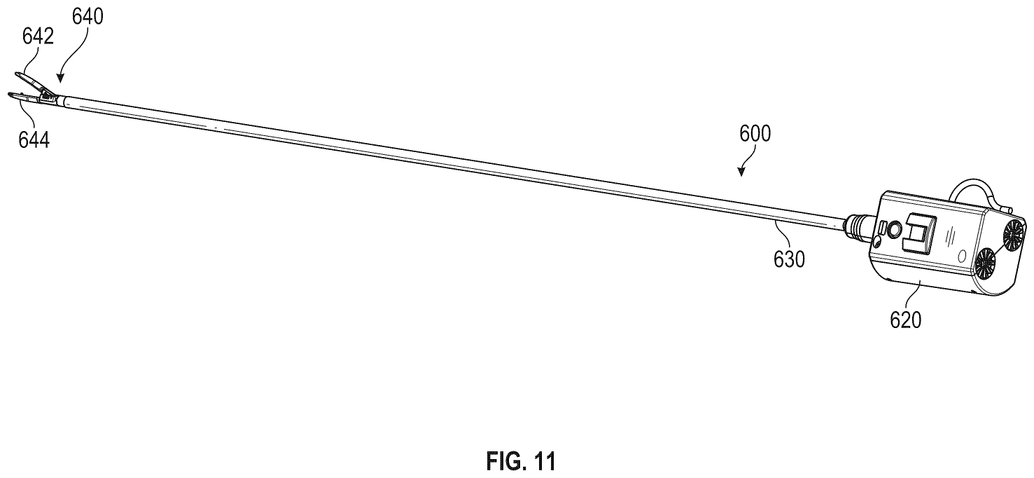

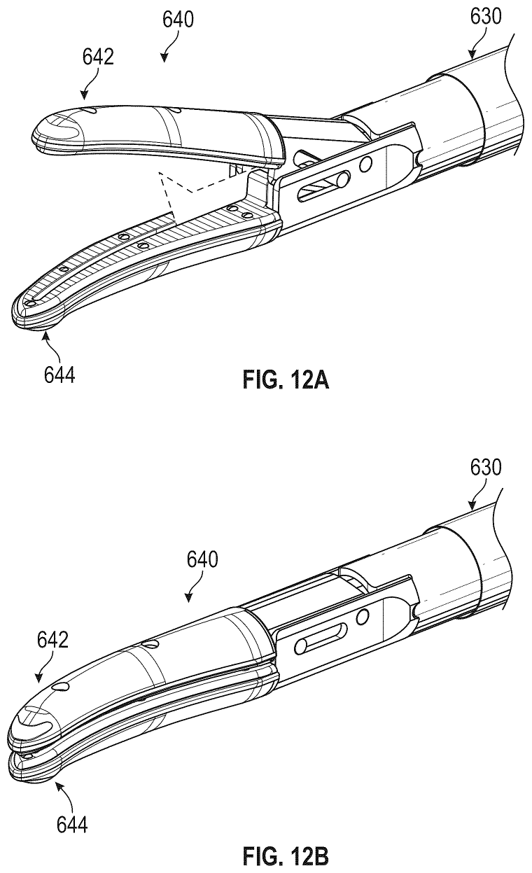

The disclosed system and method of controlled tissue sealing may be applied to any tissue scaling jawed instrument, such as an instrument 600 of FIG. 11, which is similar to the instrument 50 with some variations, such as lack of any articulation joints. This configuration minimizes the number of couplers that are being used and engaged by the IDU 52. With reference to FIGS. 11, 12A, and 12B, the instrument 600 includes a housing 620, a shaft 630 extending distally from housing 620, and end effector assembly 640 extending distally from shaft 630. The end effector assembly 640 also includes first and second jaw members 642 and 644, with the jaw member 642 being movable while the jaw member 644 is stationary relative to the shaft 630. The jaw member 642 may be actuated by a coupler (not shown) disposed at the distal end portion of the housing 620. For a more detailed description of the components of the instrument 600 and its operation reference may be made to U.S. Pat. No. 10,722,295, filed on Jan. 20, 2016, titled “Robotic surgical assemblies and electrosurgical instruments thereof,” the entire contents of which are incorporated by reference herein.

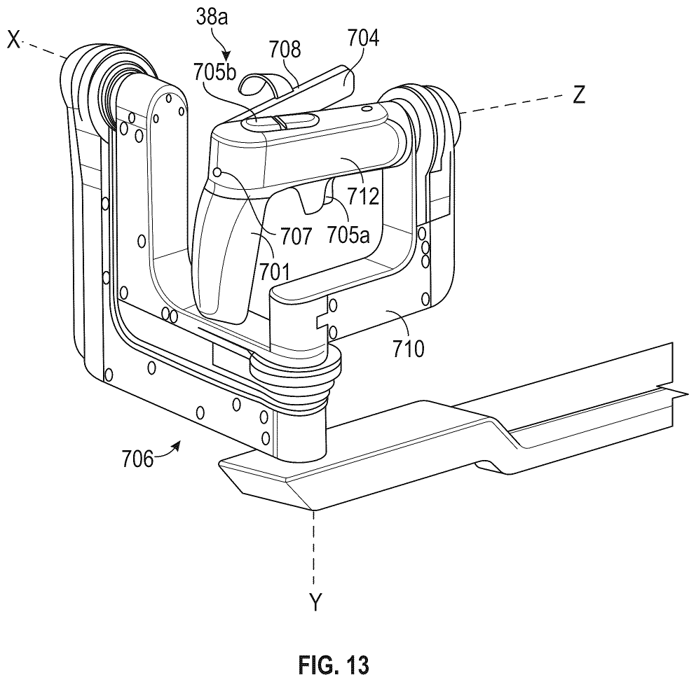

FIG. 13 shows the left-handle controller 38a, which is a mirror copy of the right-handle controller 38b. Each of the handle controllers 38a and 38b includes a handle 701 and a paddle 708 that is pivotally coupled to the handle 701 at one end (e.g., proximal) of the paddle 708. The paddle 708 is configured to control actuation, namely, opening and closing jaw members 142, 144 of the end effector assembly 140. The paddle 708 may include a finger sensor 704 configured to detect presence or movement of a finger, such as touch sensors, capacitive sensors, optical sensors, and the like. In embodiments, the finger sensor 704 may be disposed on any portion of the handle controllers 38a and 38b. Each of the handle controllers 38a and 38b may also include a trigger 705a and one or more buttons 705b for activating various functions of the instrument 50. In addition, each of the handle controllers 38a and 38b may include a gimbal assembly 706 allowing for movement and rotation of the handle controllers 38a and 38b about three axes (x, y, z). The handle controllers 38a and 38b may also include an infrared proximity sensor 707 configured to detect hand contact with a grip of the handle controllers 38a and 38b. The controller 31a of the surgeon console 30 monitors operator interactions with the handle controllers 38a and 38b and controls the instrument(s) 50 in response to operator inputs.

The paddle 708 is maintained, i.e., biased, in an open position by a feedback motor 712, which receives operator mechanical input, i.e., as the motor 712 is back driven during closure of the paddle 708 toward the closed position. The motor 712 also provides force feedback to the paddle 708 by counteracting operator's input, i.e., the motor 712 is forward driven. In addition, the motor 712 also measures the force, angle relative to the handle 701, and/or velocity of the paddle 708. The angle of the paddle 708 relative to the handle 701 is proportional to the angle between jaw members 142, 144. Thus, the paddle 708 and the jaw members 142, 144 may be fully aligned when in fully open and fully closed position and the jaw angle in between those position corresponds the paddle angle during the travel of the paddle 708.

In addition, the controller 31a also monitors individual or a new velocity of each joint of the gimbal assembly 706 as well as displacement of each of the joint of the gimbal assembly 706 and/or net displacement of the gimbal assembly 706. Details of the handle controllers 38a and 38b are provided in U.S. Patent Publication No. 2020/0315729, titled “Control arm assemblies for robotic surgical systems” filed on Nov. 30, 2018, the entire contents of which are incorporated by reference herein.

A feedback assembly 710 is disposed in the handle controller 38b to provide vibratory or haptic feedback to the operator. As shown, the feedback assembly 710 is configured to provide vibrational feedback at set frequencies and intervals to provide a sensation of touching. The feedback assembly 710 may include eccentric rotating mass (ERM) actuator, a linear resonant actuator (LRA), a piezoelectric actuator, or any other suitable tactile actuator configured to impart information to the operator through their sense of touch. Details of the haptic feedback mechanism are provided in U.S. Pat. No. 10,517,686, titled “Haptic feedback controls for a robotic surgical system interface” filed Apr. 13, 2018, the entire contents of which are incorporated by reference herein.

The paddle 708 is used to actuate various components of the instrument 50, e.g., open and close jaw members 142, 144. Thus, during use, the operator applies a force to close the jaw members 142, 144 from fully open to fully closed configuration. To maintain full jaw closure, the operator maintains force on the paddle 708 to ensure the jaw members 142, 144 are fully closed.

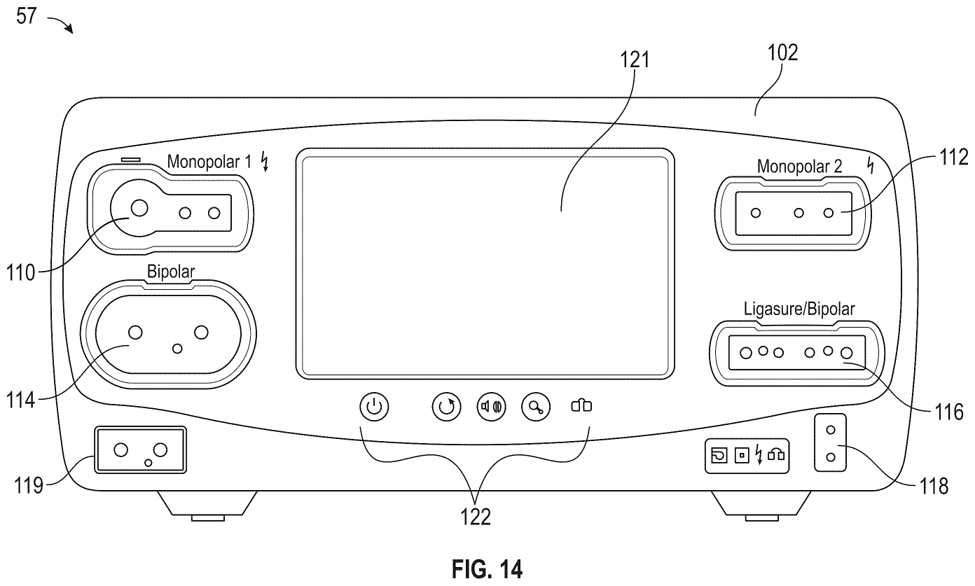

With reference to FIG. 14, a front face 102 of the generator 57 is shown. The generator 57 may include a plurality of ports 110, 112, 114, 116 to accommodate various types of electrosurgical instruments, a port 118 for coupling to a return electrode pad, and a port 119 configured to couple to a footswitch or one of the foot pedals 36, directly. The ports 110 and 112 are configured to couple to the monopolar electrosurgical instruments (e.g., monopolar electrosurgical instruments). The ports 114 and 116 are configured to couple to bipolar electrosurgical instruments (e.g., forceps and/or bipolar electrosurgical instruments 50). The generator 57 includes a display 121 for providing the user with variety of output information (e.g., intensity settings, treatment complete indicators, etc.). The display 121 is a touchscreen configured to display a menu for each of the ports 110, 112, 114, 116 and the coupled instrument. The user may also adjust inputs by touching corresponding menu options. The generator 57 may further include suitable input controls 122 (e.g., buttons, activators, switches, touch screen, etc.) for controlling the generator 57.

The generator 57 operates in a variety of modes, in which the generator 57 outputs monopolar and/or bipolar waveforms corresponding to the selected mode. Each of the modes may be activated by a button on the handle controllers 38a and 38b and/or one of foot pedals 36. Each of the modes outputs RF energy based on a preprogrammed power curve that limits how much power is output by the generator 57 at varying impedance ranges of the load (e.g., tissue). Each of the power curves includes power, voltage, and current control ranges that are defined by the user-selected intensity setting and the measured minimum impedance of the load.

The generator 57 may operate in the following monopolar modes, which include, but are not limited to, cut, blend, division with hemostasis, fulgurate, and spray. The generator 57 may operate in the following bipolar modes, including bipolar cutting, bipolar coagulation, automatic bipolar, which operates in response to sensing tissue contact, and various algorithm-controlled tissue sealing modes. The generator 57 may also output energy required to power an ultrasonic transducer, thereby enabling control and modulation of ultrasonic surgical instruments.

Each of the RF waveforms may be either monopolar or bipolar RF waveforms, each of which may be continuous or discontinuous and may have a carrier frequency from about 200 kHz to about 500 kHz. As used herein, continuous waveforms are waveforms that have a 100% duty cycle. In embodiments, continuous waveforms are used to impart a cutting effect on tissue. Conversely, discontinuous waveforms are waveforms that have a non-continuous duty cycle, e.g., below 100%. Discontinuous waveforms may be used to provide coagulation effects to tissue.

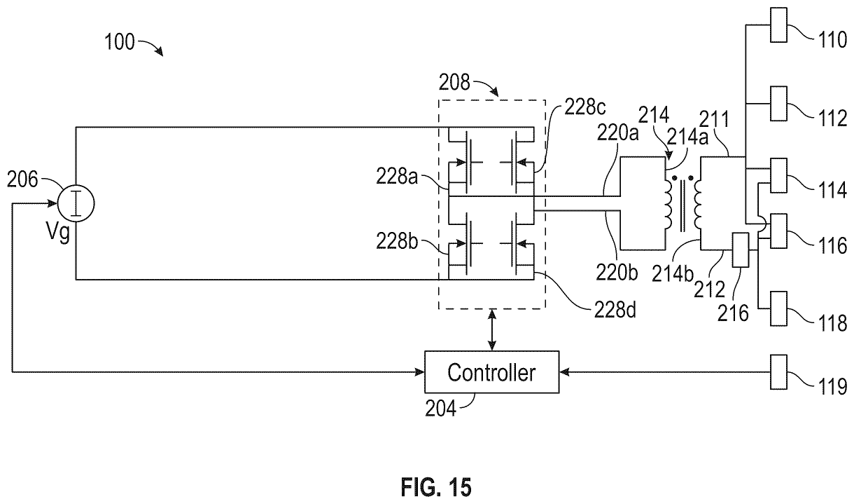

With reference to FIG. 15, the generator 57 includes a generator controller 204, a power supply 206, and an RF inverter 208. The power supply 206 may be high voltage, DC power supplies connected to a common AC source (e.g., line voltage) and provide high voltage, DC power to their respective RF inverter 208, which then convert DC power into a RF waveform through active terminal 211 and return terminal 212 corresponding to the selected mode. The active terminal 211 and the return terminal 212 are coupled to the RF inverter 208 through an isolation transformer 214. The isolation transformer 214 includes a primary winding 214a coupled to the RF inverter 208 and a secondary winding 214b coupled to the active and return terminals 211 and 212.

Electrosurgical energy for energizing the monopolar electrosurgical instruments is delivered through the ports 110 and 112, each of which is coupled to the active terminal 211. RF energy is returned through the return electrode pad coupled to the port 118, which in turn, is coupled to the return terminal 212. The secondary winding 214b of the isolation transformer 214 is coupled to the active and return terminals 211 and 212. RF energy for energizing a bipolar electrosurgical instrument is delivered through the ports 114 and 116, each of which is coupled to the active terminal 211 and the return terminal 212. The generator 57 may include a plurality of steering relays or other switching devices configured to couple the active terminal 211 and the return terminals 212 to various ports 110, 112, 114, 116, 118 based on the combination of the monopolar and bipolar electrosurgical instruments being used.

The RF inverter 208 is configured to operate in a plurality of modes, during which the generator 57 outputs corresponding waveforms having specific duty cycles, peak voltages, crest factors, etc. It is envisioned that in other embodiments, the generator 57 may be based on other types of suitable power supply topologies. RF inverter 208 may be a resonant RF amplifier or non-resonant RF amplifier. A non-resonant RF amplifier, as used herein, denotes an amplifier lacking any tuning components, i.e., conductors, capacitors, etc., disposed between the RF inverter and the load, e.g., tissue.

The generator controller 204 includes a processor operably connected to a memory and to the power supply 206 and/or RF inverter 208 allowing the processor to control the output of the RF inverter 208 of the generator 57 according to either open and/or closed control loop schemes. A closed loop control scheme is a feedback control loop, in which a plurality of sensors measures a variety of tissue and energy properties (e.g., tissue impedance, tissue temperature, output power, current and/or voltage, etc.), and provides feedback to the generator controller 204. The generator controller 204 then controls the power supply 206 and/or the RF inverter 208, which adjust the DC and/or RF waveform, respectively.

The generator 57 according to the present disclosure may also include a plurality of sensors 216, each of which monitors output of the RF inverter 208 of the generator 57. The sensors 216 may be any suitable voltage, current, power, and impedance sensors. The sensors 216 are coupled to leads 220a and 220b of the RF inverter 208. The leads 220a and 220b couple the RF inverter 208 to the primary winding 214a of the transformer 214. Thus, the sensors 216 are configured to sense voltage, current, and other electrical properties of energy supplied to the active terminal 211 and the return terminal 212.

In further embodiments, the sensor 216 may be coupled to the power supply 206 and may be configured to sense properties of DC current supplied to the RF inverter 208. The generator controller 204 also receives input (e.g., activation) signals from the display 121, the input controls 122 of the generator 57, the instruments, and/or handle controllers 38a and 38b. The generator controller 204 adjusts power output by the generator 57 and/or performs other control functions thereon in response to the input signals.

The RF inverter 208 includes a plurality of switching elements 228a-228d, which are arranged in an H-bridge topology. In embodiments, RF inverter 208 may be configured according to any suitable topology including, but not limited to, half-bridge, full-bridge, push-pull, and the like. Suitable switching elements include voltage-controlled devices such as transistors, field-effect transistors (FETs), combinations thereof, and the like. In embodiments, the FETs may be formed from gallium nitride, aluminum nitride, boron nitride, silicon carbide, or any other suitable wide bandgap materials.

The controller 204 is in communication with the RF inverter 208, and in particular, with the switching elements 228a-228d. Controller 204 is configured to output control signals, which may be pulse-width modulated (“PWM”) signals, to switching elements 228a-228d. In particular, controller 204 modulates a control signal supplied to switching elements 228a-228d of the RF inverter 208. The control signal provides PWM signals that operate the RF inverter 208 at a selected carrier frequency. Additionally, controller 204 calculates power characteristics of the RF inverter 208 output and controls the output of the generator 57 based at least in part on the measured power characteristics, which include, but are not limited to, voltage, current, and power at the output of RF inverter 208.

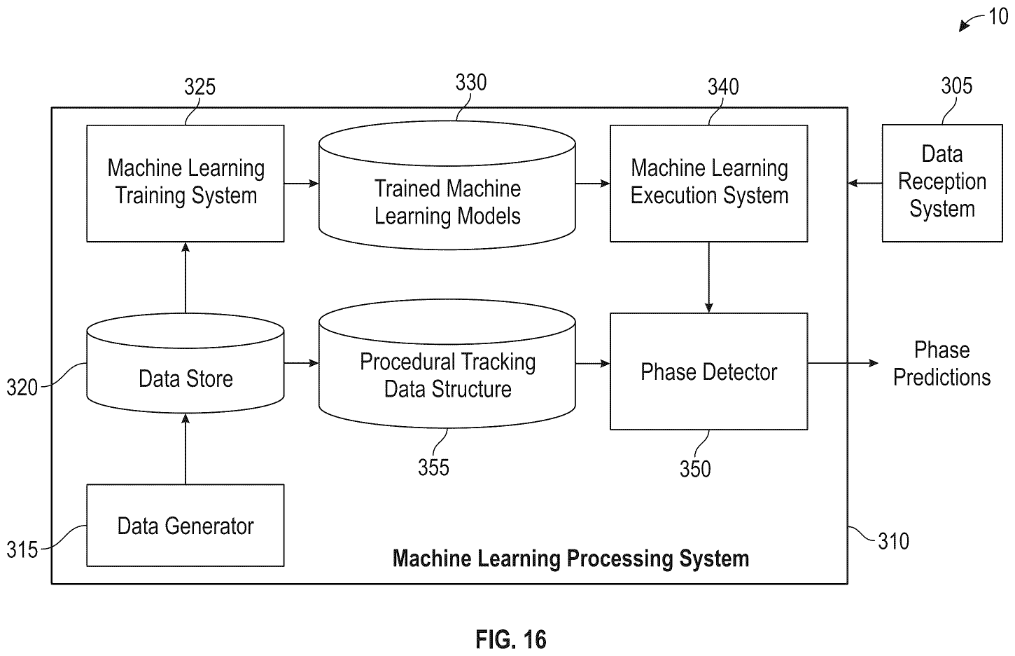

With reference to FIG. 16, the surgical robotic system 10 may include a machine learning (ML) processing system 310 that processes the surgical data using one or more ML models to identify one or more features, such as surgical phase, instrument, anatomical structure, etc., in the surgical data. The ML processing system 310 includes a ML training system 325, which may be a separate device (e.g., server) that stores its output as one or more trained ML models 330. The ML models 330 are accessible by a ML execution system 340. The ML execution system 340 may be separate from the ML training system 325, namely, devices that “train” the models are separate from devices that “infer,” i.e., perform real-time processing of surgical data using the trained ML models 330.

System 10 includes a data reception system 305 that collects surgical data, including the video data and surgical instrumentation data. The data reception system 305 can include one or more devices (e.g., one or more user devices and/or servers) located within and/or associated with a surgical operating room and/or control center. The data reception system 305 can receive surgical data in real-time, i.e., as the surgical procedure is being performed.

The ML processing system 310, in some examples, may further include a data generator 315 to generate simulated surgical data, such as a set of virtual or masked images, or record the video data from the image processing device 56, to train the ML models 330 as well as other sources of data, e.g., user input, arm movement, etc. Data generator 315 can access (read/write) a data store 320 to record data, including multiple images and/or multiple videos.

The ML processing system 310 also includes a phase detector 350 that uses the ML models to identify a phase within the surgical procedure. Phase detector 350 uses a particular procedural tracking data structure 355 from a list of procedural tracking data structures. Phase detector 350 selects the procedural tracking data structure 355 based on the type of surgical procedure that is being performed. In one or more examples, the type of surgical procedure is predetermined or input by user. The procedural tracking data structure 355 identifies a set of potential phases that may correspond to a part of the specific type of surgical procedure.

In some examples, the procedural tracking data structure 355 may be a graph that includes a set of nodes and a set of edges, with each node corresponding to a potential phase. The edges may provide directional connections between nodes that indicate (via the direction) an expected order during which the phases will be encountered throughout an iteration of the surgical procedure. The procedural tracking data structure 355 may include one or more branching nodes that feed to multiple next nodes and/or may include one or more points of divergence and/or convergence between the nodes. In some instances, a phase indicates a procedural action (e.g., surgical action) that is being performed or has been performed and/or indicates a combination of actions that have been performed. In some instances, a phase relates to a biological state of a patient undergoing a surgical procedure. For example, the biological state may indicate a complication (e.g., blood clots, clogged arteries/veins, etc.), pre-condition (e.g., lesions, polyps, etc.). In some examples, the ML models 330 are trained to detect an “abnormal condition,” such as hemorrhaging, arrhythmias, blood vessel abnormality, etc.

The phase detector 350 outputs the phase prediction associated with a portion of the video data that is analyzed by the ML processing system 310. The phase prediction is associated with the portion of the video data by identifying a start time and an end time of the portion of the video that is analyzed by the ML execution system 340. The phase prediction that is output may include an identity of a surgical phase as detected by the phase detector 350 based on the output of the ML execution system 340. Further, the phase prediction, in one or more examples, may include identities of the structures (e.g., instrument, anatomy, etc.) that are identified by the ML execution system 340 in the portion of the video that is analyzed. The phase prediction may also include a confidence score of the prediction. Other examples may include various other types of information in the phase prediction that is output. The predicted phase may be used by the controller 21a to determine when to enable controlled tissue sealing mode.

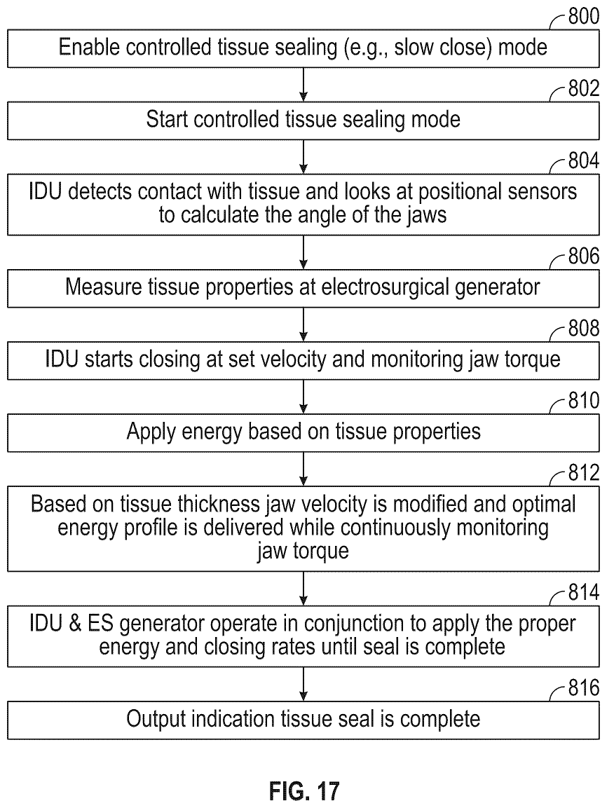

A method for controlled tissue scaling is shown in FIG. 17 and may be implemented as software instructions stored in non-transitory storage accessible and executable by a processor (e.g., controller 21a). Although specific component controllers (e.g., IDU controller 41d, generator controller 204, etc.) are described below, any controller of the system 10 may be used to control the operation of the components described below.

Initially, at step 800, the controlled tissue sealing mode is enabled, which may be done automatically by the system 10, manually by the user, or a combination of the two. The phase detector 350 may determine the phase of the procedure at which the controlled tissue scaling mode (hereinafter “the mode”) is enabled. The phase may be detected using the video feed of the camera 51 and other data provided to the system 10. In embodiments, the user may enable the mode via the GUI shown on the second screen 34 of the surgeon console 30. In further embodiments, rather than automatically enabling the mode, the controller 21a may output a prompt on the GUI asking the user whether the mode should be enabled after the phase detector 350 determined the mode may be used at a specific point of the procedure.

System 10 may also be operated in a manual mode, where the user approximates the paddle 708 manually at a desired rate of speed and the IDU 52 controls approximation of the jaw members 142, 144 based on the approximation rate of the paddle 708. Once the jaw members 142, 144 are fully closed based on the manual input, the user may then activate the electrosurgical generator 57 via a press of one of the foot pedals 36 and/or on of the buttons 705b. Following delivery of electrosurgical energy, the user may press the trigger 705a to advance a knife to cut the scaled tissue. As described below, these steps, or at least the closure and energy delivery steps, are automated in response to a single activation of a user input, e.g., button or foot pedal.

At step 802, once the mode is enabled, the system 10 enters the mode, which may be done in response to user input through the handle controller 38a or 38b, one of the foot pedals 36, and/or a GUI button. Any one of the following input mechanisms, such as trigger 705a, buttons 705b, or the paddle 708, may be used to activate the mode by either pressing and holding or pressing and releasing the desired actuation mechanism. Once the mode is started, the controller 21a controls operation of the IDU 52 and the electrosurgical generator 57 automatically without user intervention, i.e., manually closing the paddle 708 at a desired closure rate. The paddle 708 may be fully approximated at any suitable speed to reach the fully closed position relative to the handle 701. Thus, the rate at which the jaw members 142, 144 are closed is controlled by the IDU controller 41d regardless of the closure rate of the paddle 708. The paddle 708 may be maintained in the closed position until the tissue seal is completed. In this mode, the controller 21a, upon receiving the command, controls both the IDU 52, via the IDU controller 41d, to close the jaw members 142 and 144 and the electrosurgical generator 57, via the generator controller 204, to output RF energy to the jaw members 142 and 144.

At step 804, once the mode is commenced, the IDU controller 41d approximates the jaw members 142 and 144 until tissue contact is detected based on increase in torque applied by the motors 152a-d based on the feedback from the torque sensor 155. The IDU controller 41d also calculates the jaw angle between the jaw members 142 and 144 by converting the measured motor position from the sensors 157. The jaw members 142 and 144 may be closed until a desired force threshold is reached at which the IDU controller 41d may measure the jaw angle, which may be used to determine tissue thickness. This value may be used along with electrical properties to adjust compression of tissue and delivery of electrosurgical energy to the tissue.

At step 806, which may be performed in parallel with step 804, the electrosurgical generator 57 measures electrical properties of the tissue contacting the jaw members 142 and 144, and generally electrical properties of the RF energy delivered to the tissue (e.g., tissue impedance, tissue temperature, output power, current and/or voltage, etc.). The electrical properties may be used as initial parameters for determining energy delivery output.

At step 808, the IDU controller 41d commences the jaw closure process according to a preset closure algorithm. The closure algorithm may include controlling the motors 152a-d to close the jaw members 142 and 144 at a constant velocity, i.e., closure rate of the jaw angle, constant force, or combination thereof at different stages of the closure process. The velocity may be preset for each instrument 50 as a default velocity and may be adjustable by the user and/or the system via the phase detector 350, which adjusts the initial preset velocity automatically. Thus, initially the closure rate may start at a first rate and may then be adjusted continuously based on tissue thickness, which is based on jaw angle (i.e., measured via motor position) and/or measured force, which is provided by motor torque.

At step 810, which may be performed in parallel with step 808, the electrosurgical generator 57 outputs RF energy based on the measured tissue and/or energy parameters according to an energy delivery algorithm. Various algorithms may be used to control delivery of RF energy to the tissue. One exemplary energy delivery algorithm may adjust output to achieve a desired end impedance threshold indicative of seal completion where the threshold is calculated based on the initial measured impedance.

At step 812, during the compression process, velocity or other closure parameter may be modified based on measured tissue parameter such as thickness (e.g., jaw angle), pressure applied to the tissue, etc. The IDU controller 41d may monitor torque of the motors 152a-d to determine whether tissue is ripping, which may be done based on change in rate of jaw torque. In response to detecting ripping or other decrease in tissue integrity, the IDU controller 41a may pause and/or slow down velocity to prevent tissue ripping.

Simultaneously with control of the closure of the jaw members 142 and 144, the RF output of the electrosurgical generator is also adjusted based on the changes in electrical properties of the tissue and/or energy delivered thereto. Electrical properties change in response to continual delivery of RF energy, e.g., increase in impedance as tissue is desiccating and/or coagulating, as well as changes in mechanical properties, e.g., becoming denser due to compression.

At step 814, the controller 21a determines whether the tissue seal is complete. Tissue seal is deemed compete upon reaching both the desired mechanical tissue parameter (e.g., tissue pressure and/or thickness) and electrical parameter (e.g., tissue impedance). The closure process may continue until an ending threshold in jaw angle and/or force is reached. Thus, the compression may continue until the tissue is compressed to a set thickness, i.e., jaw angle, and/or achieving a desired pressure on the tissue. The pressure threshold at completion of the compression process may be from about 3 kg/cm² to about 16 kg/cm² and, in embodiments, from about 7 kg/cm² to about 13 kg/cm². Delivery of energy may continue until electrical parameters are satisfied, as well as other conditions, e.g., dwell time for parameter at that threshold, etc.

At step 816, the controller 21a outputs an indication that the tissue seal is completed. The indication may be a prompt on the GUI of the screen 32 as well as a haptic feedback indication via the handle controller 38a or 38b. The controller 21a may also output an indication that the tissue seal failed if either the compression or RF energy delivery processes did not reach the desired completion thresholds. The failure message may include specific reasons, such as pressure and/or tissue thickness (i.e., jaw angle) not reached, tissue impedance not reached, etc.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A surgical robotic system comprising:
a robotic arm including:
an instrument drive unit; and
an instrument coupled to and actuatable by the instrument drive unit, the instrument including a first jaw member and a second jaw member, wherein at least one of the first jaw member or the second jaw member is movable from an open jaw position to a closed jaw position;
an electrosurgical generator for outputting electrosurgical energy through the first and second jaw members;
a surgeon console for receiving user input to initiate a tissue sealing mode; and
a controller for simultaneously controlling the electrosurgical generator and the instrument drive unit in response to the user input to initiate the tissue sealing mode, wherein the controller commands the electrosurgical generator to output the electrosurgical energy and the instrument drive unit to close at least one of the first jaw member or the second jaw member at a set closure rate to form a tissue seal.

2. The surgical robotic system according to claim 1, wherein the instrument drive unit includes a motor for actuating at least one of the first jaw member or the second jaw member.

3. The surgical robotic system according to claim 2, wherein the instrument drive unit includes a motor sensor for measuring at least one motor parameter.

4. The surgical robotic system according to claim 3, wherein the motor sensor is a torque sensor for measuring motor torque.

5. The surgical robotic system according to claim 4, wherein the controller calculates pressure applied by the first and second jaw members based on the motor torque and determines whether the tissue seal is complete based on the calculated pressure.

6. The surgical robotic system according to claim 3, wherein the motor sensor is a position sensor for measuring angular position of the motor.

7. The surgical robotic system according to claim 6, wherein the controller calculates a jaw angle between the first and second jaw members based on the angular position of the motor and determines whether the tissue seal is complete based on the calculated jaw angle.

8. The surgical robotic system according to claim 1, wherein the electrosurgical generator includes an electrical sensor for measuring at least one parameter of the electrosurgical energy.

9. The surgical robotic system according to claim 8, wherein the controller determines whether the tissue seal is complete based on a comparison of the at least one parameter of the electrosurgical energy to a threshold.

10. A method for controlling a robotically assisted tissue sealing instrument, the method comprising:
receiving user input to start a tissue sealing mode;
controlling an instrument drive unit in response to the user input to initiate the tissue sealing mode, wherein the instrument drive unit is coupled to an instrument including a first jaw member and a second jaw member, such that at least one of the first jaw member or the second jaw member is movable from an open jaw position to a closed jaw position by the instrument drive unit; and
controlling an electrosurgical generator to output electrosurgical energy through the first and second jaw members in response to the user input to initiate the tissue sealing mode, wherein the instrument drive unit and the electrosurgical generator are operated simultaneously to compress tissue disposed between the first and second jaw members and supply the electrosurgical energy to the tissue form a tissue seal.

11. The method according to claim 10, wherein controlling the instrument drive unit further includes controlling a motor for actuating at least one of the first jaw member or the second jaw member.

12. The method according to claim 11, further comprising:
measuring at least one motor parameter at a motor sensor operatively coupled to the motor.

13. The method according to claim 12, wherein measuring the at least one motor parameter further includes measuring motor torque.

14. The method according to claim 13, further comprising:
calculating pressure applied by the first and second jaw members based on the motor torque; and
determining whether the tissue seal is complete based on the calculated pressure.

15. The method according to claim 12, wherein measuring the at least one motor parameter further includes measuring angular position of the motor.

16. The method according to claim 15, further comprising:
calculating a jaw angle between the first and second jaw members based on the angular position of the motor; and
determining whether the tissue seal is complete based on the calculated jaw angle.

17. The method according to claim 10, further comprising:
measuring at least one parameter of the electrosurgical energy at an electrical sensor operatively coupled to the electrosurgical generator.

18. The method according to claim 17, further comprising:
determining whether the tissue seal is complete based on a comparison of the at least one parameter of the electrosurgical energy to a threshold.

19. A surgical robotic system comprising:
a robotic arm including:
an instrument drive unit including a motor and a drive sensor for measuring a mechanical parameter; and
an instrument coupled to and actuatable by the instrument drive unit, the instrument including a first jaw member and a second jaw member, wherein at least one of the first jaw member or the second jaw member is movable from an open jaw position to a closed jaw position;
an electrosurgical generator for outputting electrosurgical energy through the first and second jaw members, the electrosurgical generator including a generator sensor for measuring an electrosurgical energy parameter;
a surgeon console receiving user input to initiate a tissue sealing mode from at least one of a foot pedal, a handle controller, or a screen displaying a graphical user interface; and
a controller for simultaneously controlling the electrosurgical generator and the instrument drive unit in response to the user input to initiate the tissue sealing mode, wherein the controller commands the electrosurgical generator to output the electrosurgical energy and the instrument drive unit to close at least one of the first jaw member or the second jaw member at a set closure rate to form a tissue seal.

20. The surgical robotic system according to claim 19, wherein the controller further determines whether the tissue seal is formed based on the electrosurgical energy parameter reaching an energy parameter threshold and a mechanical parameter reaching a mechanical threshold.
